(19) United States
(12) Patent Application Publication
(10) Pub. No.: US 2025/0134708 A1
(43) Pub. Date: May 1, 2025

(54) APPARATUS FOR PROVIDING COMPRESSION IN OPHTHALMIC SURGICAL CASSETTES AND METHODS OF USE THEREOF

(71) Applicant: Alcon Inc., Fribourg (CH)

(72) Inventors: Steven Benjamin ROLL, San Clemente, CA (US); Behzad HASHEMI, Irvine, CA (US)

(21) Appl. No.: 18/911,530

(22) Filed: Oct. 10, 2024

Related U.S. Application Data
(60) Provisional application No. 63/594,699, filed on Oct. 31, 2023.

(51) Int. Cl. A61F 9/007; A61M 1/00

(52) U.S. Cl. CPC A61F 9/007; A61M 1/72

(57) ABSTRACT

Embodiments herein provide a surgical cassette and surgical console for ophthalmic irrigation or aspiration during a surgical procedure, the surgical cassette comprising a plurality of valve assemblies, wherein each valve assembly comprises a retaining ring coupled to a base of the surgical cassette, and a valve body disposed within a cavity defined within the retaining ring, wherein the retaining ring provides a compression force against the valve body that in turn compresses at least a portion of a sealing material disposed on the valve body against a surface of the base. The surgical console comprises a plurality of valve drive assemblies configured to apply additional compression forces to the valve assemblies of the surgical cassette. The valve drive assemblies may be actuated individually or collectively so as to provide additional compression to selected ones of the plurality of valve assemblies.

### CROSS-REFERENCE TO RELATED APPLICATIONS

This application claims benefit of and priority to U.S. Provisional Application Ser. No. 63/594,699 (filed on Oct. 31, 2023), the content of which is incorporated herein by reference in its entirety.

### INTRODUCTION

Cataract surgery involves removing a cataractous lens and replacing the lens with an artificial intraocular lens (IOL). The cataractous lens is typically removed by fragmenting the lens and aspirating the lens fragments out of the eye. The lens may be fragmented using, e.g., a phacoemulsification probe, a laser probe, or another suitable instrument. During the procedure, the probe fragments the lens, and the fragments are aspirated out of the eye through, e.g., a hollow needle or cannula. Throughout the procedure, irrigating fluid is pumped into the eye to maintain an intraocular pressure (IOP) and prevent collapse of the eye.

During cataract surgery, a surgical cassette having one or more peristaltic and/or venturi pumps and one or more valve assemblies may be operably coupled with a fluidics module of a surgical console to facilitate the aspiration and irrigation functionalities described above. In general, the one or more valve assemblies of the surgical cassette are operable to control the application of pressure and vacuum generated by the one or more pumps during the surgical procedure.

However, conventional surgical cassettes have a number of significant shortcomings including, for example, providing insufficient compression forces between a sealing material disposed on a valve and an internal surface within the surgical cassette over time, thereby leading to an inability to maintain a robust seal for each valve within the surgical cassette, among others.

Therefore, there is a need for improved surgical cassettes which address at least some of the drawbacks described above.

### BRIEF SUMMARY

In certain embodiments, a surgical cassette is provided for ophthalmic irrigation or aspiration during a surgical procedure, the surgical cassette comprising a plurality of valve assemblies, wherein each valve assembly comprises a retaining ring coupled to a base of the surgical cassette, and a valve body disposed within a cavity defined within the retaining ring, wherein the retaining ring provides a compression force against the valve body that in turn compresses at least a portion of a sealing material disposed on the valve body against a surface of the base. In addition to the retaining ring applying a compression force to the valve body, the surgical console also applies a compression force to the same valve assemblies, according to certain embodiments. By providing a compression force from the surgical console instead of or in addition to the compression forces provided by each retaining ring, the materials comprising each valve assembly undergo less mechanical stress over time and therefore have a longer period of active use. Additionally, since at least a portion of the compression forces required to make a seal are provided by the surgical console, each valve assembly within the surgical cassette can be comprised of more common materials as opposed to more expensive specialty materials that are configured to maintain compression on their own.

In certain embodiments, a system for providing compression from a surgical console to a surgical cassette during a surgical procedure is provided, the system comprising at least one valve assembly disposed within the surgical cassette, at least one valve drive assembly disposed within the surgical console configured to engage a valve body within the at least one valve assembly, and a clamp assembly disposed within the surgical console configured to apply a distally directed force to the surgical cassette, wherein the at least one valve drive assembly is further configured to apply a force to the at least one valve assembly in response to the distally directed force applied by the clamp assembly to compress a sealing material disposed on a first end of the valve body.

In certain embodiments, a method is provided for a surgical console to provide a proximally directed force to a surgical cassette during an ophthalmic procedure, the method comprising coupling the surgical cassette to the surgical console, engaging a valve drive assembly within the surgical console with a valve assembly within the surgical cassette, applying a reaction force from the valve drive assembly to a valve body within the valve assembly, and then compressing a scaling material disposed on a first end of the valve body.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1A illustrates an example of an ophthalmic surgical system that may be used to perform ophthalmic procedures on an eye, according to certain embodiments.

FIG. 1B is an example of subsystems of a console of the ophthalmic surgical system of FIG. 1A, according to certain embodiments.

FIG. 2A is a back side isometric view of an example surgical cassette which may be operably coupled to a console of the ophthalmic surgical system of FIG. 1A, according to certain embodiments.

FIG. 2B is a back side elevation view of the surgical cassette of FIG. 2A, according to certain embodiments.

FIG. 3 is an exploded perspective view of the surgical cassette of FIGS. 2A and 2B illustrating the components comprising each of the valve assemblies and each of the pump assemblies disposed in the surgical cassette, according to certain embodiments.

FIG. 4A is an enlarged exploded front side isometric views of a portion of the surgical cassette of FIG. 2A and 2B illustrating an example valve assembly having two passages in the valve body, according to certain embodiments.

FIG. 4B is an enlarged exploded back side isometric view of a portion of the surgical cassette of FIG. 2A illustrating an example valve assembly having two passages in the valve body, according to certain embodiments.

FIG. 5 illustrates a frontal perspective view of the fluidics subsystem of the ophthalmic surgical console of FIG. 1A, the fluidics subsystem comprising a clamp assembly and a plurality of valve drive assemblies, according to certain embodiments.

FIG. 6 is a schematic illustrating a plurality of the valve drive assemblies disposed within the fluidics subsystem of FIG. 5 applying a reactive compression force to the valve assemblies within the surgical cassette of FIGS. 2A-3, according to certain embodiments.

FIG. 7 is a schematic illustrating a plurality of valve drive assemblies disposed within the surgical console of FIG. 1A applying a compression force provided by a driver to the valve assemblies within the surgical cassette of FIGS. 2A-3, according to certain embodiments.

FIG. 8 is a schematic illustrating a plurality of valve drive assemblies disposed within the surgical console of FIG. 1A applying a compression force provided by a plate to the valve assemblies within the surgical cassette of FIGS. 2A-3, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

The present disclosure relates generally to ophthalmic surgical cassettes, surgical consoles, and methods of use thereof.

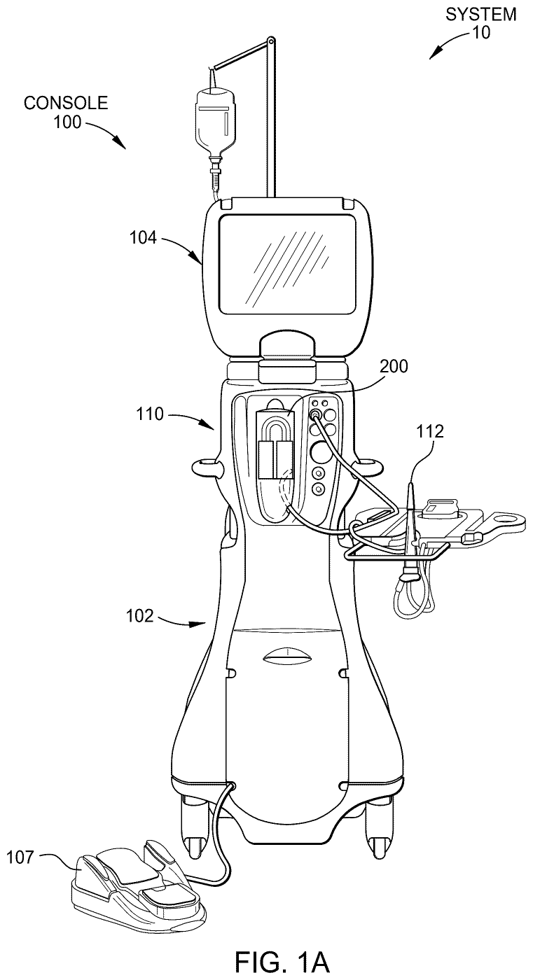

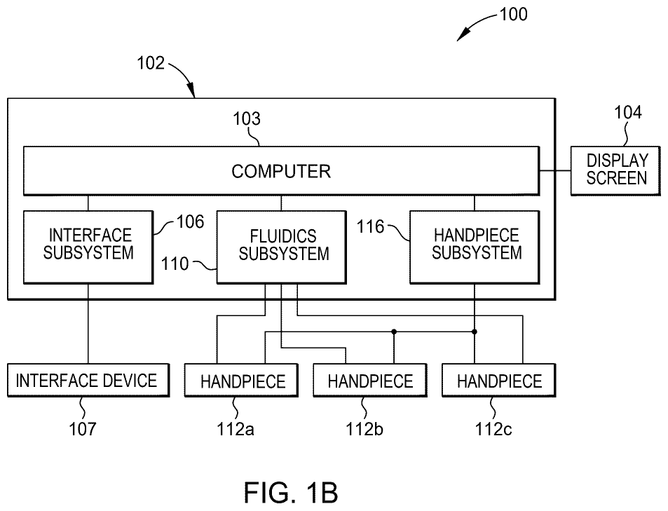

FIG. 1A illustrates an example of an ophthalmic surgical system 10 that may be used to perform ophthalmic procedures on an eye, according to certain embodiments. In the illustrated embodiments, surgical system 10 includes surgical console 100 (also referred to as a “surgical console”), a housing 102, a display screen 104, an interface device 107 (e.g., a foot pedal), a fluidics subsystem 110, and handpieces 112 (112a-c), coupled as shown and described in more detail with reference to FIG. 1B.

FIG. 1B is an example of subsystems of surgical console 100 of ophthalmic surgical system 10 of FIG. 1A, according to certain embodiments. Surgical console 100 includes housing 102, which accommodates a computer 103 (with an associated display screen 104) and subsystems 106, 110, and 116, which support interface device 107 and handpieces 112. An interface device 107 receives input to surgical system 10, sends output from surgical system 10, and/or processes the input and/or output. Examples of an interface device 107 include a foot pedal, manual input device (e.g., a keyboard), and a display. Interface subsystem 106 receives input from and/or sends output to interface device 107.

Handpiece 112 may be any suitable ophthalmic surgical instrument, e.g., an ultrasonically-driven phacoemulsification (phaco) handpiece, a laser handpiece, an irrigating cannula, a vitrectomy handpiece, or another suitable surgical handpiece. Fluidics subsystem 110 provides fluid control for one or more handpieces 112 (112a-c). For example, fluidics subsystem 110 may manage fluid for an irrigating cannula. Handpiece subsystem 116 supports one or more handpieces 112. For example, handpiece subsystem 116 may manage ultrasonic oscillation for a phaco handpiece, provide laser energy to a laser handpiece, control operation of an irrigating cannula, and/or manage features of a vitrectomy handpiece.

Computer 103 controls operation of ophthalmic surgical system 10. In certain embodiments, computer 103 includes a controller that sends instructions to components of surgical system 10 to control the surgical system 10. A display screen 104 shows data provided by computer 103.

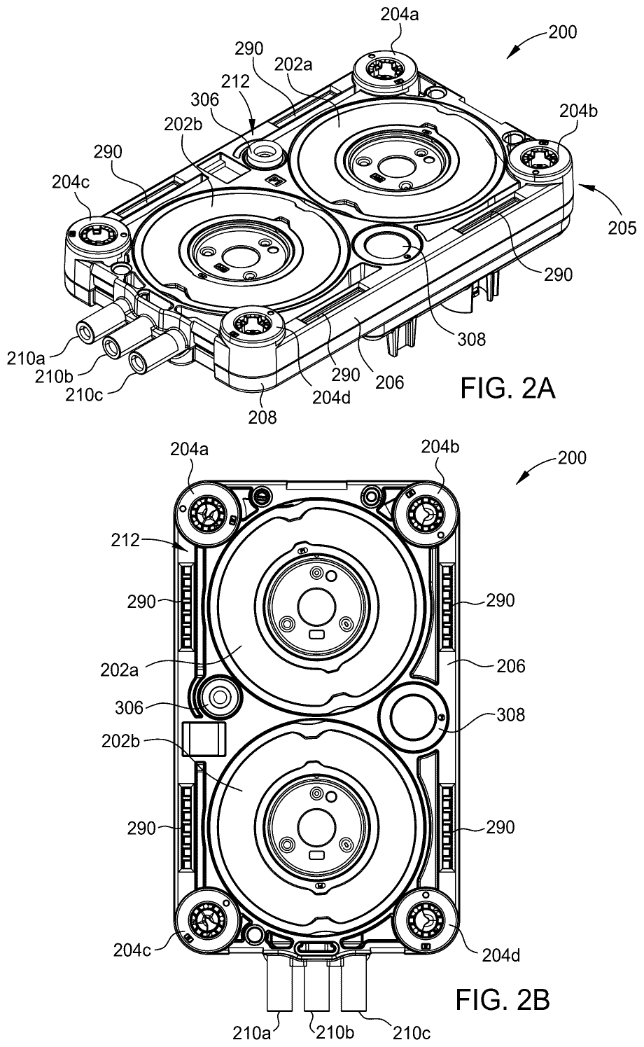

FIG. 2A is a back side isometric view of an example surgical cassette 200 which may be operably coupled to a console of an ophthalmic surgical system (e.g., surgical console 100 of ophthalmic surgical system 10 illustrated in FIGS. 1A-1B), according to certain embodiments. FIG. 2B is a back side elevation view of surgical cassette 200 of FIG. 2A, according to certain embodiments. FIGS. 2A-2B are described together herein for clarity. Surgical cassette 200 includes two pump assemblies 202 (202a-b) which provide a source of pressure and/or vacuum and four valve assemblies 204 (204a-d, also referred to as “valves”) which control pressure and/or fluid communication within surgical cassette 200. In certain other embodiments, there may be only one pump assembly or more than two pump assemblies. In certain other embodiments, there may be more or less than four valve assemblies (e.g., two to six valve assemblies).

In certain embodiments, an external source of pressure and/or vacuum is coupled to surgical cassette 200. In such embodiments, the external source may be either in place of or in addition to pump assemblies 202.

Surgical cassette 200 has a housing 205 including a base 206, a cover assembly 208 coupled to base 206, and inlet/outlet ports 210 (210a-c) in base 206 which provide pressure and/or fluid communication between inside and outside of the housing 205. In some embodiments, flow lines (e.g., tubing) may be coupled between each port 210a-c and a corresponding component of fluidics subsystem 110 and/or a corresponding handpiece 112a-c (shown in FIGS. 1A-1B).

In certain embodiments, one of a first pump assembly 202a or second pump assembly 202b provides a source of pressure (e.g., to create a driving force for fluid irrigation) while the other one of the first pump assembly 202a or second pump assembly 202b provides a source of vacuum (e.g., to create suction for fluid aspiration). The first pump assembly 202a and second pump assembly 202b may be peristaltic pumps or any other suitable type of pump for generating pressure and/or vacuum. In certain embodiments, the first pump assembly 202a and second pump assembly 202b are identical to each other.

Valve assemblies 204 are coupled to base 206. Valve assemblies 204 function cooperatively to control pressure and/or fluid communication within and through surgical cassette 200. In the illustrated embodiments, surgical cassette 200 includes a first valve assembly 204a, a second valve assembly 204b, a third valve assembly 204c, and a fourth valve assembly 204d. As shown, in the embodiments of FIG. 2A, the four valve assemblies 204 are arranged at the four corners of housing 205 and surrounding the two pump assemblies 202 which are arranged towards a center of housing 205. However, in certain other embodiments, pump assemblies 202 and valve assemblies 204 may have any other suitable arrangement.

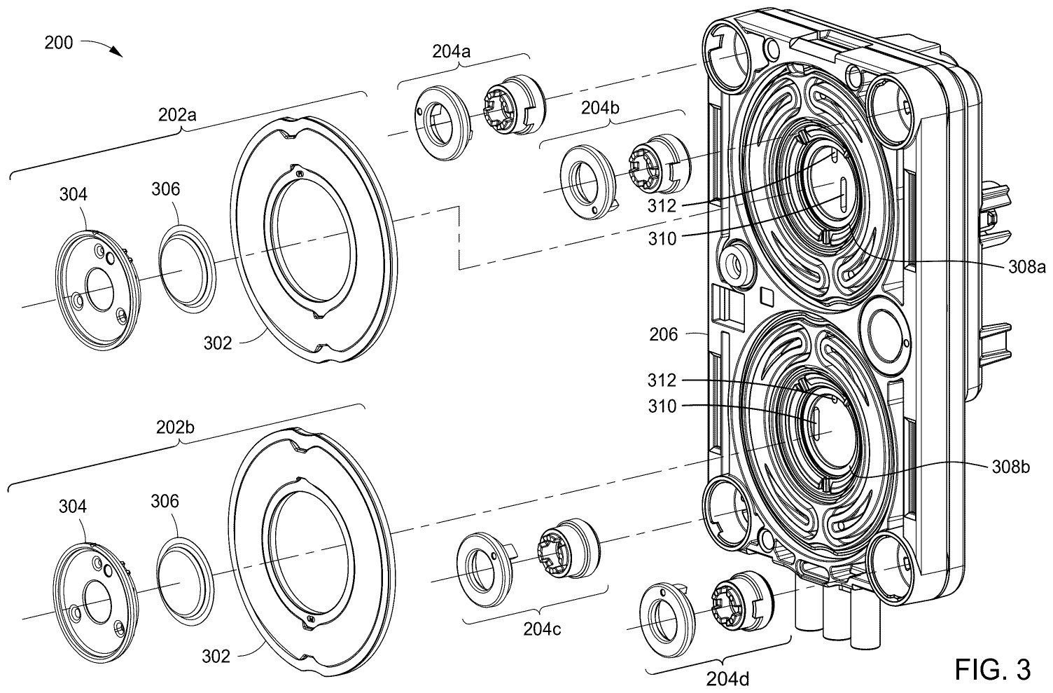

As seen in the exploded view of FIG. 3, each pump assembly 202a, 202b is coupled to and disposed within a first well 308a and a second well 308b, respectively, that are defined within the base 206. Each well 308a, 308b comprises an inlet 310 and an outlet 312 defined therein, the inlet 310 and outlet 312 being in fluid communication with the internal channels disposed within the surgical cassette 200. Each pump assembly 202a, 202b comprises a pump elastomer 302 which is disposed around an outer circumference of each well 308a, 308b. Disposed in a center portion of each well 308a, 308b in a substantially nested or stacked configuration is a diaphragm 306 which is accommodated or disposed below a diaphragm retainer ring 304. In certain embodiments, the diaphragm retainer ring 304 is coupled to the internal surfaces of the well 308a, 308b through an ultrasonic welding process with the diaphragm 306 disposed underneath. The diaphragm retainer ring 304 thereby maintains the diaphragm 306 within each respective well 308a, 308b and providing a hermetic seal with the base 206.

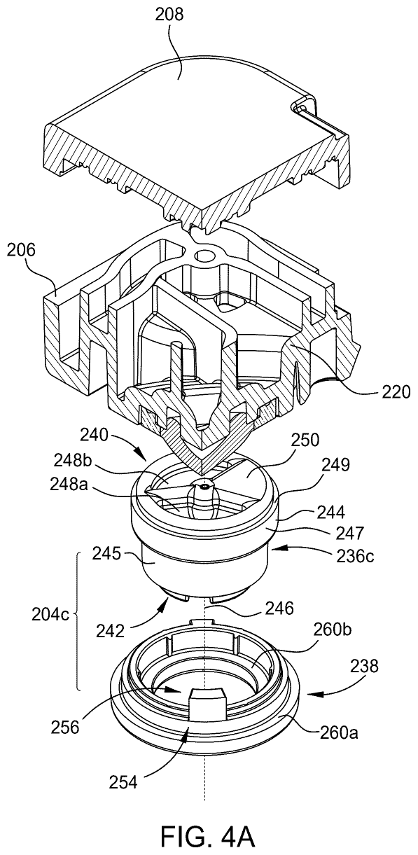

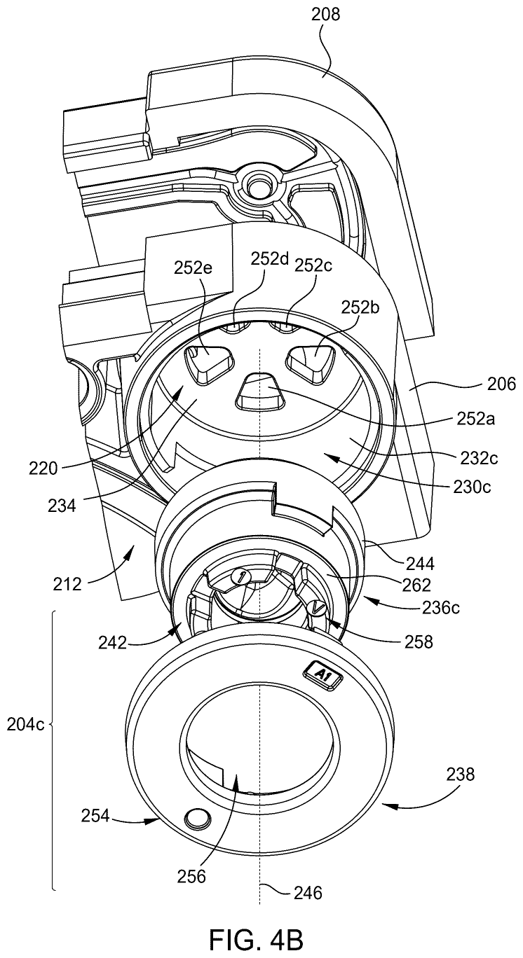

Greater detail of the valve assemblies 204 and the operation thereof within the base 206 may be seen in FIGS. 4A and 4B, which show a top down perspective exploded view and a bottom up perspective exploded view of the third valve assembly 204c, respectively. Each valve assembly 204 generally includes a valve body 236 configured to be disposed in a corresponding bore 230 and a retaining ring 238 for retaining the valve body 236 in the bore 230. The valve body 236 and retaining ring 238 fit together in a stacked arrangement. The valve body 236 is disposed between back side surface 234 and the corresponding retaining ring 238. The retaining ring 238 applies a retention force on the corresponding valve body 236 to press the valve body 236 against back side surface 234, as described in more detail below. In some other embodiments, instead of being defined within base 206, each bore 230 may be defined within a corresponding retaining ring 238 that fits around a corresponding valve body 236. In some other embodiments, each valve body 236 may be rotatably coupled to base 206 using a retention cap. The retention cap may be disposed through valve body 236 (e.g., aligned with a longitudinal axis of valve body 236). In such embodiments, each valve body 236 may be coupled to base 206 without being disposed in a corresponding bore 230.

Valve body 236 of third valve assembly 204c as seen in FIGS. 4A and 4B has a first end 240, a second end 242, a cylindrical outer surface 244 connecting first end 240 and second end 242, and a longitudinal axis 246 orthogonal to first end 240. Cylindrical outer surface 244 includes multiple stepped portions having different outer dimensions. In some embodiments, the cylindrical outer surface 244 is comprised of a base portion 245 separated from a collar portion 247, the collar portion 247 having a larger diameter relative to the base portion 245 as best seen in FIG. 4A. In one embodiment, the base portion 245 has an approximate diameter of 13 mm (millimeters) and a longitudinal length of 5 mm, while the collar portion has an approximate diameter of 16 mm and a longitudinal length of 3 mm. Transitioning from the collar portion 247 to a sealing material 250 is a shoulder 249.

Valve body 236 is rotatable about longitudinal axis 246. In certain embodiments, two passages 248 (248a-b) are formed in valve body 236 at first end 240, while in other embodiments one passage may be formed in each valve body 236. In FIG. 4A, first passage 248a and second passage 248b are about equal in length when measured in a circumferential direction about longitudinal axis 246 (e.g., extending about longitudinal axis 246 in a circumferential direction by about 140° (degrees) to 150°). In the illustrated embodiments, each passage 248 is sized to simultaneously open fluid communication with two ports of base 206 as described in more detail below respect to FIGS. 4A-4B. In some other embodiments, each passage 248 may be sized to simultaneously open fluid communication with any suitable number of ports (e.g., two, three, or four ports). In the illustrated embodiments, passages 248 include arc-shaped annular segments extending circumferentially about longitudinal axis 246. In certain embodiments, a cross-section of passages 248 may be circular, round, oval, polygonal, square, any other suitable shape, or combinations thereof. Terminal ends of each passage 248 are defined through first end 240 of valve body 236. In certain embodiments, a center axis of each passage 248 at the terminal ends is parallel to longitudinal axis 246. In certain embodiments, at least a portion of each passage 248, e.g., the portion between the terminal ends, is orthogonal to longitudinal axis 246. In certain embodiments, during fabrication, passages 248 are machined or molded in a direction parallel to longitudinal axis 246, e.g., starting from first end 240. In other words, an entire surface of each passage 248 is visible from first end 240 when viewed in a direction parallel to longitudinal axis 246. In the illustrated embodiments, passages 248 include an equal or symmetrical flow area. In some other embodiments, passages 248 may have different or asymmetric flow areas. In certain other embodiments, there may be only one passage or more than two passages formed in the valve body.

Valve body 236 includes a sealing material 250 at first end 240 which rotatably contacts back side surface 234 of base 206 for sealing first end 240 with back side surface 234. Sealing between first end 240 and back side surface 234 forms a sealing interface between planar (e.g., non-cylindrical) surfaces. Because the scaling interface is on a longitudinal end (i.e., first end 240) of valve body 236, this sealing arrangement may be referred to as end-sealing or as a “face seal.” In certain embodiments, sealing material 250 is formed from a rubber or elastomeric material (e.g., silicone rubber) which is bonded (e.g., overmolded) on valve body 236 at first end 240. In some other embodiments, valve body 236 and sealing material 250 may be integrally formed from the same material (e.g., high density polyethylene).

Retaining ring 238 has an annular body 254 with a center opening 256. Retaining ring 238 fits over and around valve body 236 such that a drive interface 258 (shown in FIG. 4B, also referred to as a “drive receiver”) of valve body 236 is received within center opening 256. In some embodiments, drive interface 258 engages a drive mechanism of surgical console 100 for rotating valve body 236 about longitudinal axis 246. Annular body 254 includes multiple stepped portions having different outer dimensions. At least one portion of annular body 254 is disposed radially between outer cylindrical surface 244 of valve body 236 and cylindrical inner wall 232c of bore 230c. At least another portion of annular body 254 is disposed outside bore 230c. An outer shoulder 260a formed between the stepped portions of annular body 254 contacts back side 212 of base 206 when retaining ring 238 is fully seated in bore 230c. In certain embodiments, a lubricant (e.g., silicone oil) may be used to facilitate relative rotation between interfacing surfaces of valve body 236 (e.g., back side surface 262) and retaining ring 238 (e.g., inner shoulder 260b), and/or between sealing material 250 and back side surface 234. In certain embodiments, one or more of the interfacing surfaces may be impregnated with lubricant. In some embodiments, the lubricant may be any liquid that provides a lubricious and/or hydrophobic coating.

The retention force applied by the retaining ring 238 is applied in a direction parallel to longitudinal axis 246 (axially) and forces first end 240 of valve body 236 towards back side surface 234 of base 206 which compresses sealing material 250 against back side surface 234 thereby forming a seal between passages 248 and corresponding ports 252. In certain embodiments, sealing material 250 is compressed in the direction parallel to longitudinal axis 246 (axially) up to 34% of its total height, thereby compressing or reducing the overall height down to approximately 1 mm, when retaining ring 238 is fully seated in bore 230c. In certain embodiments, retaining ring 238 is coupled to base 206 using a solid-state welding technique (e.g., ultrasonic welding) so that no gap or space is present between the inner shoulder 260b of the retaining ring 238 and the back side surface 262 of the valve body 236. In some other embodiments, retaining ring 238 may for example be snap-fit, threaded, and/or adhered to base 206. In certain embodiments, the valve body 236 itself may be snap-fit, threaded, and/or adhered directly to base 206 without use of the retaining ring 238.

In certain embodiments, valve body 236 includes a hard stop feature which can be used to correlate a rotational state of valve body 236 with one of the base 206 or retaining ring 238 in order to ensure proper alignment between passages 248 and corresponding ports 252 during operation.

In certain other embodiments, the hard stop feature may comprise one or more optical sensors or indicators disposed on the valve body 236 and the retaining ring 238, respectively. For example, instead of physical contact being made between the valve body 236 and the retaining ring 238, the one or more optical sensors may indicate when a first maximum rotational position between the valve body 236 and retaining ring 238 is achieved, thereby indicating to the system what the current alignment or rotational position the valve body 236 is in relative to the retaining ring 238 and/or to the base 206.

In certain embodiments, five ports 252 are formed through lower wall 220. In operation, valve body 236 is rotatable relative to back side surface 234 of base 206 to align each passage 248 with a corresponding port 252 (252a-e) of base 206 to open pressure and/or fluid communication between corresponding ones of the plurality of channels defined within the base 206. A flow axis through each port 252 is parallel to longitudinal axis 246 of valve body 236. A shape of each port 252 may correspond to a cross-section of each passage 248 of valve body 236 to help maintain flow therethrough. In certain embodiments, a cross-sectional shape of each passage 248 may be formed by continuing a shape of the corresponding port 252 as a swept surface through valve body 236.

In the illustrated embodiments, five ports 252 are formed through lower wall 220 within each bore 230. However, there may be any suitable number of ports (e.g., three to seven ports) in each bore 230. In the illustrated embodiments, ports 252 include arc-shaped trapezoidal segments. In some other embodiments, ports 252 may be circular, round, oval, polygonal, square, any other suitable shape, or combinations thereof. In the illustrated embodiments, ports 252 have an equal flow area. In some other embodiments, ports 252 may have different flow areas. In the illustrated embodiments, ports 252 are uniformly spaced in the circumferential direction. In some other embodiments, ports 252 may have different spacing in the circumferential direction.

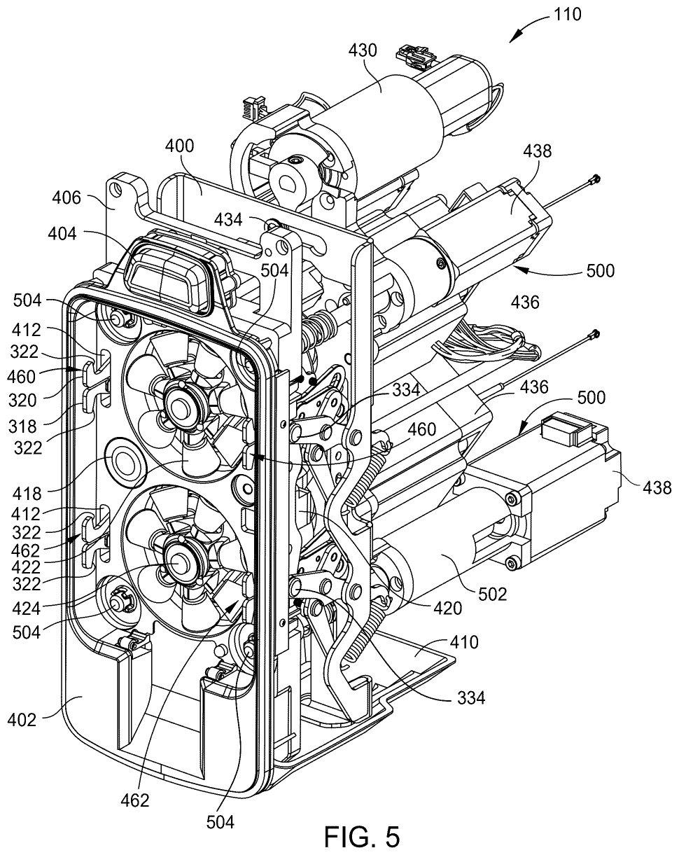

In certain embodiments, a clamp assembly 400 is incorporated into the fluidics subsystem 110 of the surgical console 100, as shown in FIG. 5. The clamp assembly 400 is disposed on a proximal or frontal portion of the fluidics subsystem 110, near a front surface of the surgical console 100. In certain embodiments, the clamp assembly 400 is disposed behind a face plate 406 of the fluidics subsystem 110 and over a drain pan 410 coupled to the face plate 406. The clamp assembly 400 is coupled to the face plate 406 by a base pin 334, which is inserted through each of an upper and a lower clamp mechanism 460, 462 of clamp assembly 400 and terminates within a corresponding aperture within a side portion or edge of the face plate 406. The face plate 406 comprises a plurality of slots 412 defined through its surface, which in certain embodiments, are symmetrically defined vertically and horizontally about the face plate 406. When the fluidics subsystem 110 is assembled, the clamp assembly 400 and face plate 406 are aligned with one another so that hooks 322 and distal portions of a first arm 318 and a second arm 320 of the upper and lower clamp mechanisms 460, 462 are disposed through and extend out of a corresponding slot 412. Each slot 412 is large enough to permit relative vertical movement of the hooks 322, as well as a degree of horizontal movement therein. In certain embodiments, a pressure sensor 418 and a camera 420 are further disposed through the face plate 406 for monitoring the performance of a surgical cassette when attached. In certain embodiments, the camera 420 is configured to read any barcodes on the surgical cassette 200.

In certain embodiments, a bezel 402 is coupled to the frontal surface of the face plate 406 as shown in FIG. 5. The bezel 402 has an opening or aperture defined therein which is large enough to dispose the surgical cassette 200 there through in order to removably couple to the exposed face plate 406. A button 404 disposed on a top portion of the bezel 402 may be used to actuate the clamp assembly 400 as is further detailed below.

In certain embodiments, a motor plate is disposed distally behind the clamp assembly 400. A motion plate motor 430 and a plurality of pump motors 436 are coupled to the motor plate. In certain embodiments, the motion plate motor 430 comprises a rotating motor pin 434 that is inserted through the motion plate. The pump motors 436 are coupled to the motor plate and extend though the interior of the motion plate of the clamp assembly 400 so as to engage each of a plurality of hub roller assemblies 422 disposed in the face plate 406 which in turn serve to circulate or move fluid through the surgical cassette 200 when attached. Disposed at or near each hub roller assembly 422 is an additional pressure sensor 424 for monitoring the pressure within the cassette.

In certain embodiments, a plurality of valve drive assemblies 500 are disposed within the fluidics subsystem 110, each valve drive assembly 500 comprising a valve drive motor 438, a valve drive shaft 502 coupled to a proximal portion of the valve drive motor 438, and a valve drive head 504 in turn coupled to a proximal end of the valve drive shaft 502. Each valve drive head 504 is configured to extend or be disposed through the clamp assembly 400, while the valve drive motor 438 of each valve drive assembly 500 remains distal of the clamp assembly 400. The proximal most portion of each valve drive head 504 is inserted through the face plate 406 and is left exposed to engage and actuate the corresponding plurality of valve assemblies 204 that are disposed within the surgical cassette 200 as detailed below.

In certain embodiments, the surgical cassette 200 is first inserted into the fluidics subsystem 110 by orientating it over the face plate 406 with the upper and lower clamp mechanisms 460, 462 in the “closed” position, namely wherein the first and second arms 318, 320 are orientated so that the respective hooks 322 are adjacently disposed to each other and face opposing directions. When each clamp mechanism 460, 462 is in the “closed” position, this allows the surgical cassette 200 to be disposed or slipped over the opposing hooks 322, namely by disposing a corresponding plurality of slots 290 seen in FIGS. 2A and 2B defined within the surgical cassette 200 over each of the hooks 322 so that the hooks 322 enter an internal portion of the surgical cassette 200.

In certain embodiments, at the same time the clamp mechanisms 460, 462 are inserted into the surgical cassette 200, each of the valve assemblies 204 and the pump assemblies 202 of the surgical cassette 200 are pressed against and then engaged with the corresponding plurality of valve drive heads 504 and the plurality of hub roller assemblies 422, respectively. For example, each of the valve drive heads 504 are inserted through the annular body 254 of the retaining ring 238 and then coupled to or are otherwise engaged with the drive interface 258 disposed on the second end 242 of the valve body 236 of the corresponding valve assembly 204. Each hub roller assembly 422 in turn is pressed against and makes contact with the pump elastomer 302 corresponding to each pump assembly 202a, 202b. With each of the valve drive heads 504 engaged with a valve body 236 of a corresponding valve assembly 204, the valve motor 438 is activated which begins to rotate the valve drive shaft 502 in either a clockwise or counterclockwise direction relative to the valve body 236. The valve drive shaft 502 likewise rotates the valve drive head 504 in the same rotational direction which causes the valve body 236 to rotate within the valve assembly 204, thereby opening, closing, or otherwise manipulating the fluid flow paths communicated to each valve assembly 204. In this manner, the valve assemblies 204 within the surgical cassette 200 may be actuated by the valve drive assemblies 500 within the fluidics subsystem 110 so as to provide the required aspiration or irrigation functions during an ophthalmic procedure.

In certain embodiments, when the clamp assembly 400 is actuated, the hooks 322 of each of the upper and lower clamp mechanisms 460, 462 expand or open within the slots 290 defined within the surgical cassette 200 in which it is disposed and into a hollow interior or cavity defined therein. The angled ends of the hooks 322 ensure that a portion of each hook 322 extends through the surgical cassette 200 and remains there, thereby preventing or at least minimizing any distal movement of the surgical cassette 200 relative to the surgical console 100. The hooks 322 further push the cassette against the face plate 406, which ensures that engagement between the valve drive heads 504 and the valve assemblies 204 of the surgical cassette 200 are maintained while also ensuring that the sealing material 250 disposed on the first end 240 of each valve body 236 is adequately compressed against the corresponding back side surface 234 of each respective bore 230. According to certain embodiments, there are at least four valve assemblies 204 within the surgical cassette 200, each of which provides functional modalities by closing and opening different fluid channels within the surgical cassette 200. Each valve body 236 within each valve assembly 204 comprises a soft sealing material 250, molded as a second shot to a rigid substrate material. The sealing material 250 is compressed to maintain a seal within the surgical cassette 200 at least in part by the retaining ring 238. However, there are challenges associated with providing and maintaining valve compression. Namely, viscoelastic behavior, also known as stress relaxation and/or creep, of the elastomer and other rigid polymers on the surgical cassette 200, valve bodies 236, and retaining rings 238 occurs after prolonged shelf life, which ultimately decreases valve compression and/or compression force. To combat these losses and possible loss of seal, a targeted amount of compression must be applied to each valve body 236 which compensates for compression-loss over time and for variances in part manufacturing tolerances. Compression and/or compression force of the sealing material 250 is critical as it directly correlates to the torque required to rotate the valve bodies 236 within the surgical cassette 200. Specifically, when the sealing material 250 is compressed, a higher reaction force is provided by the ballooning of the sealing material 250 into ports 252, thereby creating greater resistance to rotation.

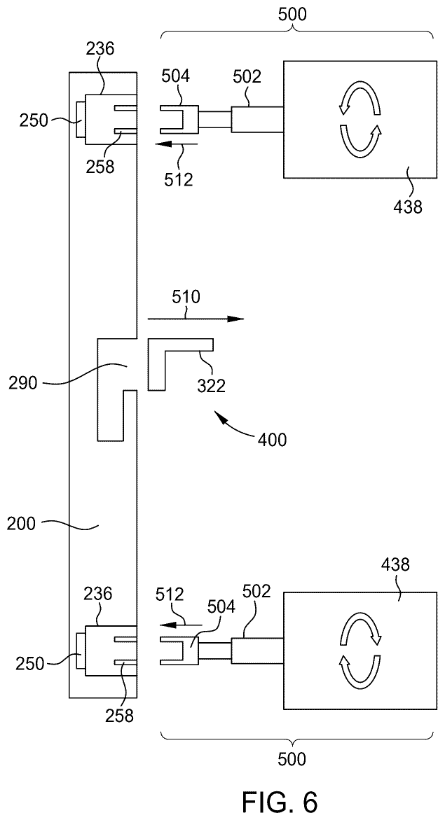

The current embodiment aims to decrease the amount of valve compression needed during assembly of the surgical cassette 200 by relying on the surgical console 100 to apply valve compression, as opposed to the retaining ring 238. By providing a compression force from the surgical console 100, the current embodiment avoids applying high reaction forces on the valve assemblies 204 by the retaining rings 238 over the life of the surgical cassette 200. Applying high reaction forces on the valve assemblies 204 by the retaining rings as used in previous designs requires the retaining ring 238 to apply increased valve compression so as to compensate for loss of compression over time, manufacturing tolerances, and/or part tolerances. Additionally, high reaction forces on the valve assemblies 204 leads to a higher torque being required to rotate the valve assemblies 204 as well as stress relaxation of materials used within the valve assemblies 204. These issues in turn require a stronger ultrasonic weld to be used between the retaining ring 238 and the surgical cassette 200 so as to withstand such reaction forces. For example, the ultrasonic welding process used to couple the retaining ring 238 to the surgical cassette 200 requires increased force and custom equipment to compensate for the loss in compression over time. However, if the ultrasonic weld is applied incorrectly, this can lead to too much compression and abrasion during rotation of the valve assemblies 204. Additionally, sufficient compression provided by the retaining ring 238 can be difficult to achieve unless using “specialty materials” given the high reaction forces present within the valves. For example, the retaining ring 238 must be comprised of specialty materials capable of withstanding high reaction forces on the valve assembly 204 over time, thereby increasing the overall cost of the surgical cassette. Therefore, according to certain embodiments, in addition to the retaining rings 238 of the valve assemblies 204, the valve drive assembly 500 of the current embodiments provides additional means for ensuring sufficient or appropriate compression of each valve assembly 204 for firm sealing thereof, according to certain embodiments as illustrated in FIGS. 6-8.

In the example of FIG. 6, a fixed-valve drive mechanism is shown, wherein counter-forces between the clamp assemblies 400 and valve drive assemblies 500 facilitate sufficient compression of valve assemblies 204. For example, as seen in FIG. 6, as the clamp assembly 400 of FIG. 5 is being actuated to secure the surgical cassette 200 to the surgical console 100, and upon securing the surgical cassette to the surgical console 100, each hook 322 applies a force in the distal direction as indicated by arrow 510, which pulls the surgical cassette 200 against the surgical console 100. In response, each of the valve drive heads 504 push further or harder into the respective valve bodies 236 via a reaction force indicated by arrow 512, which further compresses the sealing material 250 within each bore 230. The specific size and shape of the valve drive heads 504 and the valve drive shafts 502 will alter the amount of reaction force 512 that is applied to the valve bodies 236. For example, the relative length of the valve drive shaft 502, the length of the valve drive head 504, or the materials comprising the valve drive head 504 and valve drive shaft 502 among many other dimensions will modify or alter the reaction force 512 accordingly. Generally, however, the reaction forces 512 provided by the valve drive assemblies 500 is greater than the opposing distal forces 510 provided by the coupling of the surgical cassette 200 to the surgical console 100 via the clamp assemblies 400.

In effect, the fixed-valve drive mechanism shown in FIG. 6 applies the requisite forces to compress the sealing material 250 only during use of the surgical cassette 200, as opposed to the compression forces created by the retaining rings 238 during the lifetime of the surgical cassette 200. In some examples, as a result of compression being applied only during use of the surgical cassette 200, the fixed-valve drive mechanism enables the utilization of different materials, including different plastic materials, for components of the valve assemblies 204, which would otherwise have to be able to withstand high reaction forces from a compressed valve assembly 204 over the lifetime of the surgical cassette 200.

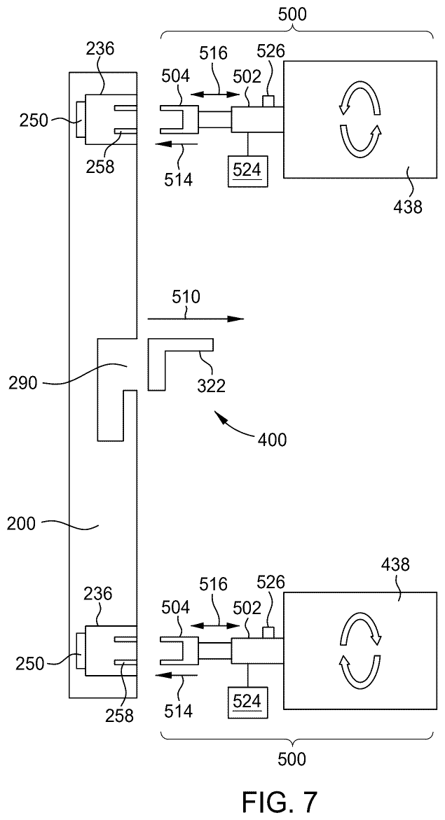

In the example of FIG. 7, a servo-driven valve drive mechanism is shown, wherein counter-forces between the clamp assemblies 400 and valve drive assemblies 500 combine with forces applied by the servo-driven valve drive mechanism to facilitate sufficient compression of valve assemblies 204. As shown, each valve drive assembly 500 in FIG. 7 comprises a driver 524 that drives bi-directional lateral and/or axial movement of the valve drive shaft 502 and/or the valve drive head 504. During operation, after the clamp assembly 400 has been actuated to secure the surgical cassette 200, each hook 322 applies a force in the distal direction as indicated by arrow 510, thereby pulling the surgical cassette 200 against the surgical console 100. In addition to the reaction forces caused by the clamp assembly 400 as discussed above, the driver 524 coupled to the valve drive assembly 500 may further actuate the valve drive shaft 502 in either the proximal or distal directions, as indicated by double sided arrow 516, to increase or decrease compression of valve assemblies 204. For example, to increase the pressure applied by the drive head 504 and further compress the sealing material 250, the driver 524 actuates the valve drive shaft 502 in the proximal direction, thereby applying a higher reaction force as indicated by arrow 514. Alternatively, if less pressure from the valve drive head 504 is desired, the driver 524 actuates the valve drive shaft 502 in the distal direction which decreases or nullifies the reaction force created in response to the actuation of the clamp assembly 400.

In certain embodiments, the valve drive assembly 500 engages the valve assemblies 204 after the clamp assembly 400 has pulled the surgical cassette 200 against the surgical console 100. In this embodiment, the forces applied by the valve drive assembly 500 are therefore independent from the counter-forces from the clamp assembly 400.

The driver 524 within the valve drive assembly 500 may comprise any suitable type of actuator or motor for driving the valve drive shaft 502 and/or the valve drive head 504, such as a servo motor, an electric motor, a hydraulic motor or piston, a pneumatic motor or piston, or any other means for providing axial movement. Because each valve drive assembly 500 comprises its own respective driver 524, each valve drive assembly 500 may be actuated independently from each other, allowing the surgical console 100 to automatically increase or decrease compression applied to selected valve assemblies 204 within the surgical cassette 200. In certain embodiments, each valve drive assembly 500 further comprises a force sensor 526 that detects and measures mechanical load, or physical stresses, on the valve drive head 504 and thus, contact and pressure between the valve drive head 504 and/or the drive interface 258 of valve bodies 236. Signals from the force sensor 526 may then be relayed to and/or used by the driver 524 to begin actuation of the valve drive shaft 502 by a predetermined amount or distance so as to create a seal by compressing the sealing material 250 a desired amount. Use of servo-driven valve drive assemblies 500 and a force sensor 526, or other device to sense contact with the valve assemblies 204, may provide additional benefits to those discussed above with regard to FIG. 7. For example, utilization of the driver 524, which is configured to apply a predetermined amount of force against the valve assemblies 204 or move the valve drive head 504 a predetermined distance, eliminates the effect of tolerances on the final compression of each valve assembly 204 and leads to more consistent compression of the sealing material 250. In addition, individual valve assemblies 204 are compressed separately, according to certain embodiments, which is advantageous for valve assemblies 204 that have differing geometries, torque requirements, or the like.

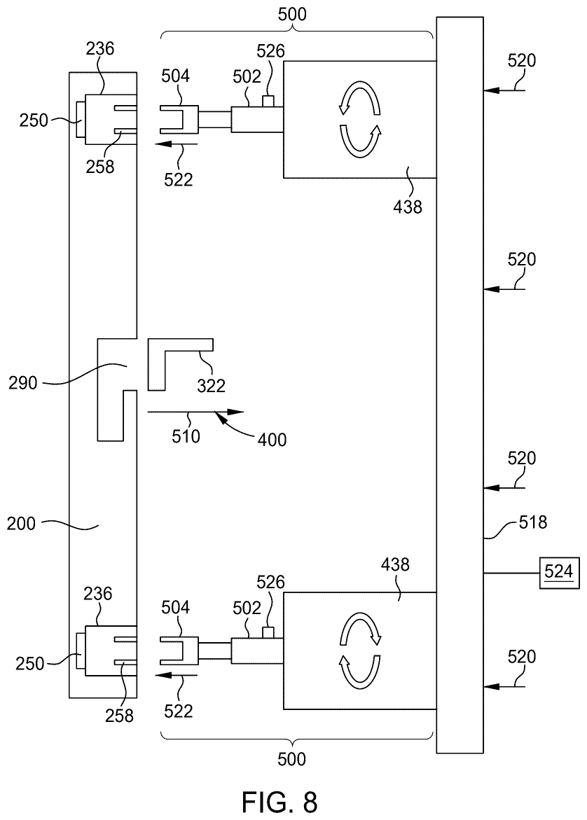

In the example of FIG. 8, a plate-driven valve drive mechanism is shown, wherein counter-forces between the clamp assemblies 400 and valve drive assemblies 500 combine with forces applied by the plate-driven valve drive mechanism to facilitate sufficient compression of valve assemblies 204. In FIG. 8, rather than coupling a driver 524 to each valve drive assembly 500, one or more drivers 524 are operably coupled to a plate 518 of the fluidics subsystem 110, and the plate 518 is further coupled to each of the valve drive assemblies 500. Accordingly, actuation of the plate 518 by the one or more drivers 524 provides the compression forces necessary to create a sufficient seal within each of the valve assemblies 204 disposed on the surgical cassette 200. For example, after the clamp assembly 400 has been actuated to secure the surgical cassette 200, each hook 322 applies a force in the distal direction as indicated by arrow 510, thereby pulling the surgical cassette 200 against the surgical console 100. In addition to the reaction forces caused by the clamp assembly 400 as discussed above, the driver 524 coupled to the plate 518 may further actuate each of the valve drive shafts 502, simultaneously, by applying a proximal force all distributed on the plate 518 (e.g., along a height and across a width of the plate 518), as indicated by arrows 520, to increase the pressure applied by each of the drive heads 504 and further compress the sealing materials 250. Alternatively, if less pressure from the valve drive heads 504 is desired, the driver 524 actuates the plate 518 in the distal direction, which can decrease or nullify the reaction force created in response to the actuation of the clamp assembly 400.

In certain embodiments, similar to FIG. 8, each valve drive assembly 500 comprises a force sensor 526 that detects contact and measures forces between the valve drive head 504 and the respective drive interface 258 of valve bodies 236, and then signals to the driver 524 to begin actuation of the plate 518 by a predetermined amount or distance so as to form a desired compression of the sealing material 250. As the plate 518 pushes proximally against each of the valve drive assemblies 500, the proximal distributed forces 520 are transferred to each of the valve drive heads 504 that, in turn, apply a proximal force against each corresponding valve body 236 as indicated by arrow 522. The force 522 provided by the valve drive head 504 pushes the valve body 236 into the surgical cassette 200 and further compresses the sealing material 250, thereby creating a robust seal between the sealing material 250 and the back side surface of the surgical cassette 200. In certain embodiments, the fluidics subsystem 110 comprises a plurality of plates 518, or a plate 518 for each one of the valve drive assemblies 500. Each of the plurality of plates 518 may be actuated separately or in unison by one or more drivers 524 so as to selectively compress one, some, or all of the valve assemblies 204 disposed within the surgical cassette 200 as is required.

As shown in FIG. 8, because each of the valve drive assemblies 500 are coupled to a common plate 518, all the valve drive assemblies 500 will move together at the same time, thereby applying the same amount of pressure to each of the valve assemblies 204 simultaneously.

Accordingly, described herein are means for providing sufficient compression between a valve drive assembly disposed within a surgical console and a valve assembly disposed within a surgical cassette, and methods of use thereof. The various embodiments as provided herein lower the amount of reaction forces experienced by the valve assembly over the life of the surgical cassette, leading to less wear and tear on the different components of the surgical cassette and the machines used during the manufacturing process. Lower reaction forces on the valve assembly leads to less valve compression being required at the time of production to compensate for losses (of compression and compression forces over-time). In certain embodiments where the drive mechanism adjusts the compression of the valve assembly after engagement, less valve compression is required at the time of production to compensate for manufacturing tolerances and/or part tolerances. Additionally, less torque is required in order to rotate the valve body of the valve assembly. Also, the chances of valve abrasion during rotation decreases due to lower compression. Furthermore, because lower reaction forces are required, less stress is applied to the cassette components including the retainer ring, base, and the joints within the surgical cassette near the valve assembly. The cassette components may therefore be formed without specialty ultrasonic weld equipment or the use of specialty materials that are normally required for a higher strength threshold or tolerance.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

What is claimed is:

1. A system for coupling a surgical console to a surgical cassette during a surgical procedure, the system comprising:
at least one valve assembly disposed within the surgical cassette;
at least one valve drive assembly disposed within the surgical console configured to engage a valve body within the at least one valve assembly; and
a clamp assembly disposed within the surgical console configured to apply a distally directed force to the surgical cassette,
wherein the at least one valve drive assembly is further configured to apply a force to the at least one valve assembly in response to the distally directed force applied by the clamp assembly to compress a sealing material disposed on a first end of the valve body, and wherein the force provided by the at least one valve drive assembly is greater than the distally directed force provided by the clamp assembly.

2. The system of claim 1, wherein the at least one valve drive assembly comprises:
a valve drive motor;
a valve drive shaft coupled to the valve drive motor; and
a valve drive head coupled to the valve drive shaft,
wherein the valve drive head is further configured to engage with a drive interface disposed on a second end of the valve body.

3. The system of claim 1, further comprising at least one driver disposed within the surgical console, the at least one driver configured to apply a proximally directed force onto the at least one valve drive assembly.

4. The system of claim 3, further comprising a plate coupled to the at least one valve drive assembly, wherein the at least one driver is configured to apply the proximally directed force onto the plate.

5. The system of claim 3, wherein the driver comprises at least one of a servo motor, a hydraulic motor, or a pneumatic motor.

6. A method for a surgical console to provide a proximally directed force to a surgical cassette during an ophthalmic procedure, the method comprising:
coupling the surgical cassette to the surgical console;
engaging a valve drive assembly within the surgical console with a valve assembly within the surgical cassette; and
applying a force from the valve drive assembly to a valve body within the valve assembly to compress a sealing material disposed on a first end of the valve body, wherein the force provided by the valve drive assembly is greater than an opposing force provided by the coupling of the surgical cassette to the surgical console via the clamp assembly.

7. The method of claim 6, wherein engaging the valve drive assembly within the surgical console with the valve assembly within the surgical cassette comprises:
aligning a valve drive head disposed on a proximal end of the valve drive assembly with the valve body; and
inserting the valve drive head into a drive interface disposed on a second end of the valve body.

8. The method of claim 6, wherein coupling the surgical cassette to the surgical console comprises applying a distally directed force from the surgical cassette to the surgical console.

9. The method of claim 8, wherein applying the force from the valve drive assembly to the valve body within the valve assembly comprises applying a reaction force in response to the distally directed force from the surgical cassette to the surgical console.

10. The method of claim 6, wherein applying the force from the valve drive assembly to the valve body within the valve assembly comprises actuating the valve drive assembly in the proximal direction.

11. The method of claim 10, wherein actuating the valve drive assembly in the proximal direction comprises actuating a plate coupled to the valve drive assembly in the proximal direction.

12. The method of claim 10, wherein actuating the valve drive assembly in the proximal direction comprises actuating at least one of a servo motor, a hydraulic motor, or a pneumatic motor coupled to the valve drive assembly.

13. The method of claim 6 further comprising:
engaging a plurality of valve drive assemblies within the surgical console with a corresponding plurality of valve assemblies within the surgical cassette; and
applying a force from each of the plurality of valve drive assemblies to a valve body within each of the plurality of valve assemblies.

14. The method of claim 13, wherein applying a force from each of the plurality of valve drive assemblies to the valve body within each of the plurality of valve assemblies comprises independently actuating each of the plurality of valve drive assemblies in the proximal direction via a driver coupled to each of the plurality of valve drive assemblies.

15. The method of claim 13, wherein applying a force from each of the plurality of valve drive assemblies to the valve body within each of the plurality of valve assemblies comprises simultaneously actuating each of the plurality of valve drive assemblies in the proximal direction via a plate coupled to each of the plurality of valve drive assemblies.
